(19) United States
(12) Patent Application Publication
(10) Pub. No.: US 2025/0329438 A1
(43) Pub. Date: Oct. 23, 2025

(54) METHOD AND SYSTEM FOR TRANSITIONING HEALTH CARE BASED ON ANALYTICS

(71) Applicants: Michelle Camicia, Novato, CA (US); Barbara J. Lutz, Chapel Hill, NC (US)

(72) Inventors: Michelle Camicia, Novato, CA (US); Barbara J. Lutz, Chapel Hill, NC (US)

(21) Appl. No.: 18/643,095

(22) Filed: Apr. 23, 2024

(51) Int. Cl. G16H 40/20; G16H 80/00

(52) U.S. Cl. CPC G16H 40/20; G16H 80/00

(57) ABSTRACT

An evidence-based assessment tool (the PATHC) is designed to assess family caregivers' preparedness to transition patients' home following inpatient medical treatment. Individualized, tailored interventions are then algorithmically selected from a Catalogue of Interventions designed to mitigate patients' risks and address unmet needs in patients', caregivers' and caregivers' plans for readiness and capacity for the role. Based on an identification of at least one specific underlying condition that was a basis for the inpatient treatment or rehabilitation hospitalization, questions relating to expected needs and abilities during post-hospitalization care are provided to at least one potential caregiver. Responses to the questions are scholastically valued, and a programmed processor executes software to determine compliance, expectations, needs and timelines to be implemented in the transition.

### GOVERNMENT LICENSE RIGHTS

Notification under obligation by virtue of 35 U.S.C. 202(c)(6), the following statement is included: This invention was made with government support under Grant No. R43 NR020679 from the National Institute of Nursing Health, a division of the National Institute of Health. The government has certain rights in the invention.

### RELATED APPLICATIONS DATA

None.

### BACKGROUND OF THE ART

### 1. Field of the Invention

The healthcare field is highly dependent upon obtaining information and appropriately using and analyzing that information to achieve most favorable outcomes for the patients, medical providers, and families of the patients. The present invention relates to a method and system for obtaining information and making clear decisions on levels of care to be provided to patients, particularly during transitional stages of treatment.

### 2. Background of the Art

Care for patients may shift in two directions, increased medical attention with decreasing health of the patient, and decreased intensity of medical intervention with achieved health improvement. The implications of the timeliness and appropriateness of these transitional steps has significant impact on the patient, the patient's family, the effectiveness of provided care and the expense of the provided care. More studies have been made in attempting to quantify analytic methodologies for determining the timing and degree of each step in these transitions. Reliance solely upon the subjective view of medical practitioners is a haphazard procedure, places significant emotional pressure on medical staff, and creates situations where tensions among the patient, patient's family and medical providers, and families of the patients. The present invention relates to a method and system for obtaining information and making clear decisions on levels of care to be provided to patients, particularly during transitional stages of treatment.

### 3. Background of the Art

Care for patients may shift in two directions, increased medical attention with decreasing health of the patient, and decreased intensity of medical intervention with achieved health improvement. The implications of the timeliness and appropriateness of these transitional steps has significant impact on the patient, the patient's family, the effectiveness of provided care and the expense of the provided care. More studies have been made in attempting to quantify analytic methodologies for determining the timing and degree of each step in these transitions. Reliance solely upon the subjective view of medical practitioners is a haphazard procedure, places significant emotional pressure on medical staff, and creates situations where tensions among the patient, patient's family and medical staff make the entire process undesirably influenced by emotions rather than medical needs.

As related in Transitional Care Management: Practical Processes for Your Practice, Neela K. Patel, et al., (https://www.aafp.org/pubs/fpm/issues/2019/0500/p27.pdf), transitional care management (TCM) covers the safe handoff of a patient from one level of care to another. During these transitions in care, the medical teams often face problems because of inaccessible (or not readily available) medical records, unclear plans on precise care changes, or limited efforts by others (particularly family) in engaging health care professionals in detailing the transition process. This analysis emphasized the need for improved communication between patients and all caregivers, as well as having well-defined protocols directing appropriate specifics in the transitions.

A study has outlined ten (10) different plans for use in determining and implementing health care transition by the Agency for Healthcare Research and Quality in their 2021 report on “Care Coordination and Care Plans for Transitions Across Care Settings.” (https://effectivehealthcare.ahrq.gov/products/care-coordination-plans/research).

Family caregivers are often required to provide post-discharge assistance to individuals with disabling conditions, resulting in strain and negative effects on caregivers' health and well-being. Recent legislation through the RAISE Family Caregivers Act (national) and CARE Act (state) has made addressing the needs of family caregivers a priority. The American Association of Retired Persons (AARP), Centers for Disease Control (CDC), and the National Academies of Science, Engineering, and Medicine (NASEM) have all developed recommendations focusing on assessing and addressing the needs of family caregivers (Camicia M, Lutz B J, Stram D, Tucker L Y, Ray C, Theodore B R. Improving Caregiver Health through Systematic Assessment and a Tailored Plan of Care. West J Nurs Res. 2021:1939459211045432).

While assessing and addressing the needs of family caregivers has been identified as a priority area, there are few validated assessment tools available and no comprehensive programs designed to fill this gap (M, Lutz B J. Implementing a Caregiver Assessment and Tailored Plan: An Emerging Case Management Competency. Prof Case Manag. 2021;26(4):205-213). There are validated tools to assess the outcomes of caregiving, e.g. the Caregiver Strain Index (Camicia M E, Ann Laslo, Robinson B C. Validation of a Caregiver Strain Index. J Gerontol. 1983;38(3):344-348; and Thornton M, Travis SS. Analysis of the reliability of the modified caregiver strain index. J Gerontol B Psychol Sci Soc Sci. 2003;58(2):S127 132) and the Bakas Caregiving Outcomes Scale (Bakas T, Champion V, Perkins S M, Farran C J, Williams L S. Psychometric testing of the revised 15-item Bakas Caregiving Outcomes Scale. Nurs Res. 2006;55(5):346-355), and tools to assess caregiver preparedness after the family member has been in the caregiver role for some time. However, there were no validated tools designed to screen family members' readiness to provide post-discharge care and no comprehensive programs that link assessment results to corresponding interventions designed to meet individual unmet caregiver needs.

Implementation of one embodiment of a process according to the present technology is shown in the flow diagram of FIG. 1. The Post-Implementation Group received all aspects of usual care, with the following additions: (1) during the admission assessment the nurse case manager obtained the email address of the designated primary caregiver; (2) approximately halfway through the expected stay at the IRF, the family caregiver was sent an email with a link to complete the PATH assessment; (3) the nurse case manager reviewed the PATH assessment results to identify items with a score of ≤2 indicating low or no preparation; (4) developed a tailored caregiver plan of care by selecting interventions that corresponded to each low scoring item from a drop down menu (Catalogue of Interventions) in the electronic health record (EHR); and (5) the nurse case manager and inter-professional team implemented the plan (FIG. 1).

### SUMMARY OF THE INVENTION

The PATH2Caregiving Program™ system and method incorporates an evidence-based assessment tool (the PATH©) that is designed to assess family caregivers' preparedness to transition patients' home following inpatient rehabilitation hospitalization. Individualized, tailored interventions are then algorithmically selected from a Catalogue of Interventions designed to mitigate the risks and address the unmet needs in caregivers' and caregivers' plans for readiness and capacity for the role. Based on an identification of at least one specific underlying condition that was a basis for the rehabilitation hospitalization, questions relating to expected needs and abilities during post-hospitalization care are provided to at least one potential caregiver. Responses to the questions are scholastically valued, and a PATH© program processor executes software to determine compliance, expectations, needs and timelines to be implemented in the transition and after transition from the hospital to the next care facility, especially to a home care environment.

### BRIEF DESCRIPTION OF THE FIGURES

FIG. 1 shows a flow diagram of a caregiving program according to the present technology.

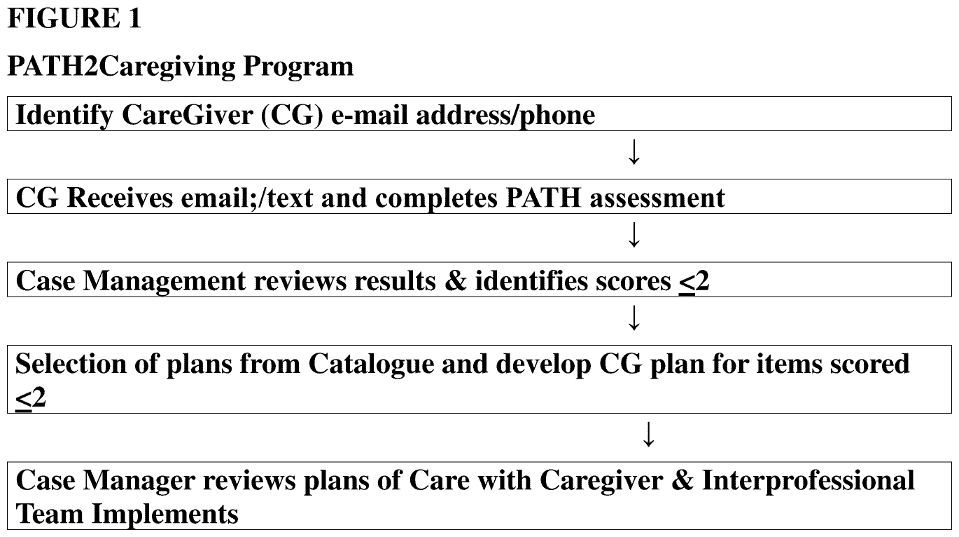

FIG. 2 shows a flow diagram for processor executed steps in implementing technology according to the present technology.

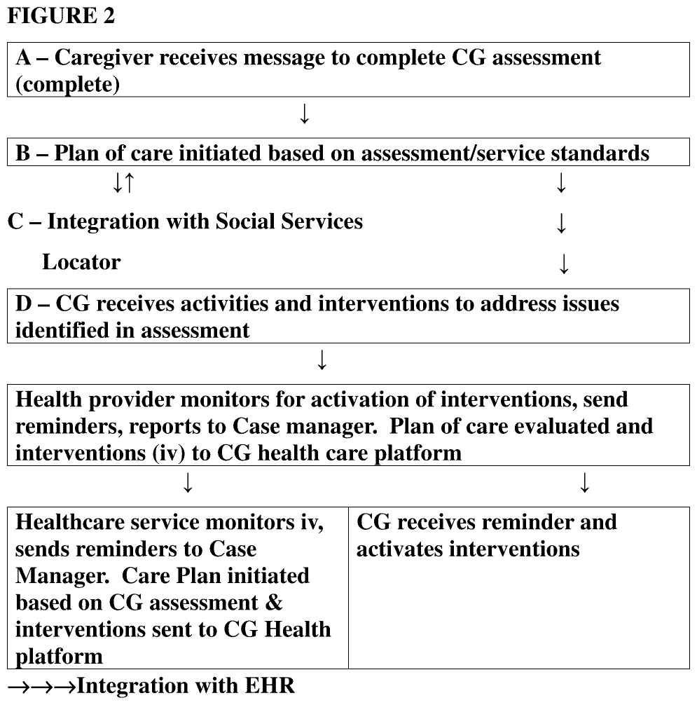

FIG. 3 shows a list of content desirably included within the evaluated parameters of the present technology.

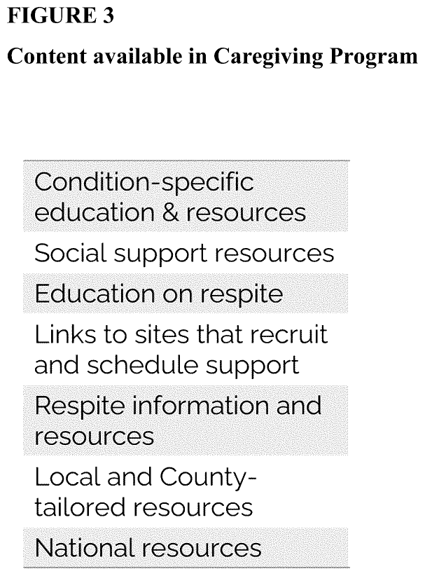

FIG. 4A, 4B, 4C and 4D show individual potential caregiver scholastic rating charts.

FIG. 5 shows a procedure for follow-up monitoring of specific patient during and after transitioning of care.

### DETAILED DESCRIPTION OF THE INVENTION

A method is provided for improving extra-institutional care for a patient by:
a) Identifying a potential caregiver for a specific patient prior to transitioning from institutional care to home care;

Because of the volume and types of personal data involved in this information, it is likely that highly secure storage and access will be implemented, with advanced encryption used to access and output data.
b) Establishing an online communication link between a central processor and a processor accessible by the potential caregiver;

The connection may be implemented by combinations of wired and wireless systems among the various processing and serving devices. The format for the information will be elected by the designer.
c) The central processor providing a set of questions to the processor accessible by the potential caregiver, the questions including queries concerning knowledge of and capability of providing the specific patient's, the questions selected from the group consisting of i) medical needs, ii) physical needs, iii) emotional needs, iv) the potential caregiver's physical ability to serve the specific patient, v) the potential caregiver's knowledgeability and comfort level in serving the specific patient, and vi) availability of at least one of equipment, location and accommodations desirable for the care of the specific patient;

Among the critical abilities upon which a caregiver should be evaluated may include the ability to manage pharmaceutical usage by the specific patient. Many patients may have a complex regimen of medicines, including volumes, frequencies, delivery methods (oral, topical, dressing changes, wound cleansing) that must be within the level of skill of the prospective caregiver. Physical tasks and abilities, including walk-assistance, wheelchair movement and control, body elevation within a bed or chair, and even partial lifting of a patient from bathroom devices must also be considered. The ability to assist in clothing changes and light household chores in support of the specific patient must also be considered.

d) The central processor receiving responses to the questions in a format that can be read as or converted to scholastic values for the responses;

By scholastic values are meant a system of hierarchal ranking not based on any absolute measurements, such as Please rate your ability to seat and elevate yourself from a toilet on a scale of 1 to 10, with 1 indicating an inability to perform the task without third party assistance and 10 indicating that the specific patient is capable of performing that task with no additional assistance, including the absence of pull bars. The values may be simplified to as few numbers as desired, such as 0—incapable, 1—capable with significant assistance, 2—capable with some assistances (including pull bar), and 3—capable of completely independent activity. The central processor stores all of the responses in a file specific o each patient.
e) The central processor executing software to characterize a level of scholastic performance within categories of the questions and responses;

The characterization may be provided in numerous available formats. Typically, a total sum of all scholastic values may be used, but this can be misleading and not provide an analysis in sufficient specific detail that can provide an authoritative for best results provision and clearance of caregivers. For example, the totaled scholastic value may be relatively high, but specific needs of the patient may not be within a desired level of skill sets needed for a specific patient. For example, a patient may have as many as 10 different drugs taken at different and various times over the course of the day. Where both a specific patient and a prospective caregiver do not have the organizational and reading skills necessary to administer the panoply of medications comfortably, even though other physical skills may be ranked high, the deficiency of pharmaceutical management would not necessarily be identified within a gross scholastic total. In that case, the needs of the specific patient with a specific potential caregiver could be overlooked. It is therefore desirable for an automated (e.g., specific distorted values from data of the specific patient and potential caregiver would be highlighted where scholastic values for each in a specific category are deficient, and/or Artificial Intelligence will review the total and individual combined values and decide paired appropriateness, or offer training for one or the other of the pair) analysis of total and individual response values. The caregiving system may also provide distal assistance. For example, based on stored knowledge of individual medicinal (pharmaceutical) needs of a specific patient, a pill box having weekly (a.m. and p.m. dosing) can be prepared and imaged. The image (with foreknowledge of the shapes, colors and dimensions of each pill) of the total distribution of pills over a timeline (single day or days or a week) can be analyzed by the central processor to assure that all medications are properly allotted within the pill box for a specific patient. Any errors would be reported back to the caregiver and/or specific patient. This analytical step may also be used as a test for prospective caregivers to test their ability to perform different required levels of skill in the teask and to provide an accurate scholastic rating.
f) The central processor selecting at least one stored caregiver plan consistent with the characterized level of scholastic performance within the categories of the questions and responses;

After matching a specific patient with one or more prospective caregivers, a plan is selected from a table of available plans based on respective needs of the specific patient and the abilities of each proposed caregiver. Based on identified skills and deficiencies, the central processor may modify plans and identify specific tasks that are included and excluded from basic plans based upon an analysis (by personnel or Artificial Intelligence) to design an appropriate, even if less than ideal, plan.
g) A case manager having access to the central computer and the at least one stored caregiver plan communicating with the potential caregiver to relay and explain details of the at least one stored caregiver plan prior to transfer of the specific patient to care by the potential caregiver. The case manager may be a live person or a virtual manager implementing communication through artificial voice messaging, artificial voice conversations, texting, emailing and the like. The case manager is typically a live person, but may be a virtual manager, operating on AI or by a preset program with fixed parameters. If operating on Artificial Intelligence, the processor responds to answers and questions of the specific patient by analytics in which the needs of the specific patient are evaluated beyond only the questions and answers from the specific patient, but also data provided from the primary care or hospital for the specific patient.

The method may have the central processor identify more than one stored caregiver plans, and rank the more than one stored caregiver plans according to closest matches with needs indicated by the scholastic values attributed to the responses of the potential caregiver. Cost variation among the plans may also be indicated.

The method may have the specific patient and/or the potential caregiver is given a written or oral examination to determine a level of skill relative to implementing and overseeing administration of prescribed medication for the specific patient.

The method may have more than stored caregiver plan offered to the specific patient, and the specific patient elects a single stored caregiver plan for use.

The method may have the central processor review caregiver policies from business entities and, based upon execution of software identifying best fits for available caregiver services, offers at least one caregiver plan to the specific patient.

The method may have the central processor offer a selection of multiple caregiver plans which are ranked according to at least one parameter selected from the group consisting of cost, relative match with patient needs, and ability of the caregiver plans to adjust over a time of treatment for the specific patient.

A system for enabling the above methods may have system include the central processor and the processor accessible by the potential caregiver linked through a wireless connection, the central processor including a memory storage element programmed to execute software performing steps c). d), e) and f).

Additionally, the plans may identify ways in which existing specific patient insurance or local (city, county, State, federal) plans may be accessed or used to pay for at least some of the costs involved in using the services of a caregiver. The plans may identify policies and programs that can be initially applied for without preexisting medical policies, and offer assistance (even fee-based) in obtaining such secondary sources of payment for a specific payment.

The method may also have the central processor review stored medical history of the specific patient and adjusts weighting of the scholastic values attributed to the responses of the potential caregiver in according with programmed importance of responses with respect to specific medical needs of the specific patient in accordance with proposed medical treatment for the specific patient.

The method may further have the at least one stored caregiver plan includes providing information relating to at least one resource selected from the group consisting of A) available government services for the specific patient, B) available support personnel, C) online or telephonic information regarding patient care, and D).

### Artificial Intelligence Support of the Caregiver Plan

### Detection Algorithm

As noted above, the system may include resident software, firmware, or embedded processing routines that are operative to analyze the output and input from the questions and responses in an effort to identify activities or responses that were induced by an events (i.e., a natural or an induced response). More specifically, these techniques/algorithms may attempt to establish with a high degree of confidence, that a detected match/mismatch of responses regarding abilities is a beneficial, neutral or harmful result and that the detected difference is not simply a subject-acceptable match/mismatch for a lower-importance caregiver activity. In varying embodiments, the detection techniques/algorithms may be performed in the analog/time domain, the digital/frequency domain, and/or may employ one or more wavelet analyses in an effort to promptly and accurately characterize any degree of appropriateness for the proposed match of a caregiver and specific patient. Additional techniques such as response gating, stimulus frequency modulation, artificial intelligence/structured machine learning, and/or ensemble approaches may also be used to make this detection more robust and/or provide a greater degree of confidence in the detection. While different detection techniques may each prove to be sufficiently effective in making this characterization, in many instances, however, detection confidence and detection speed/time are in conflict. The following will summarize analog/time domain detection techniques, digital/frequency detection techniques, and then go into further detail on wavelet-style analyses that have been found to generate more rapid responses for comparable levels of accuracy and at higher degrees of confidence.

### Analog/Time Domain Event Detection

In some embodiments, the signal processing algorithms used to recognize a natural or induced response may involve one or more analog detection techniques such as described, for example, in U.S. Pat. No. 8,343,065, issued on Jan. 1, 2013, which is incorporated by reference in its entirety. In the analog techniques, the processor may examine one or more aspects of the MMG output signal in an analog/time domain to determine if the sensed response includes signal attributes that are indicative of a response of the muscle to the stimulus. These analog aspects may include, for example, the time derivative of acceleration or the maximum amplitude of the M-wave/initial response being above a predetermined threshold. While these signal traits often have a high degree of sensitivity, they often deliver a significant number of false positives if viewed in isolation (i.e., a single spike in the waveform could just as easily be caused by a sharp bump of the operating table). As such, to provide a robust determination, multiple consecutive events need to be detected to make a final characterization. That said, in many instances ample muscle settling time must be provided between adjacent events to ensure that sequential muscle contractions do not overlap to introduce constructive or destructive signal interference in the waveform parameters, which are often dependent on absolute magnitudes or rates of change. The requirement for muscle settling time could limit the stimulation frequency to less than about 4 Hz, or even 2 Hz or less.

### Digital/Frequency Domain

In a digital context, such as described in US 2015/0051506, filed on Aug. 13, 2013, which is incorporated by reference in its entirety, the processor may convert the analog waveform into the frequency domain (e.g., via a discrete Fourier transform, or fast Fourier transform) and then compare the frequency characteristics of the MMG output signal with the known frequency of the applied stimulation to determine whether the sensed muscle responses and/or “events” were induced by the applied stimulus. While this is a more robust form of detection than simply searching for discrete analog signal characteristics, the Fourier transform necessarily requires a certain amount of accumulated data to perform the spectral decomposition. Thus, any performed analysis is necessarily occurring on buffered data and thus is delayed.

### Wavelet Analysis

As a third potential manner of detecting artificially induced environment or even muscle responses, the system may include software or firmware that performs a wavelet similarity analysis on the incoming signals. The use of wavelet signal analyses presents an improvement over the frequency-domain detection techniques as it operates on real-time data as it is received without the need to convert to the frequency domain via an FFT. Likewise, it provides a more robust characterization than simply examining discrete signal parameters (e.g., magnitude or rate of change) in isolation.

In a wavelet analysis, one or more analog wave patterns may be pre-selected as being reference “mother wavelets” that bare a resemblance to a smoothed MMG event. A filtered analog waveform in the MMG output signal may then be compared, in real time, to each mother wavelet to determine a degree of similarity between the two. If the presence of the mother wavelet is found within the analog signal, then the system may infer that an artificially induced muscular event has occurred. This is a more robust analysis than the analog method described above largely because it considers the entire wave shape rather than instantaneous parameters.

Because the responsiveness of each subject's body (and/or tissue groups) may have different dynamic properties, in some embodiments, the system may also search for the presence of different time-scaled variants of the mother wavelet within the analog signal. These variants are generally referred to as “daughter wavelets,” and are similar to the mother wavelet except in how compressed or stretched the wave is on the time-axis.

To perform this analysis, the system may first derive a plurality of “daughter wavelets” from each mother wavelet, where the daughter wavelets are each time-scaled versions of their respective mother wavelet. When analyzing an incoming wave, the examined wave may be continuously passed across each daughter wavelet to determine a respective degree of similarity between the incoming signal and each daughter wavelet (i.e., the degree of similarity being expressed in the form of a “convolution coefficient”). The convolution coefficient for each daughter wavelet may then vary with time as the examined wave passes across the daughter wavelet. This analysis may be performed, for example, using a continuous wavelet transform or discrete wavelet transform and may output a 2d matrix of convolution coefficients such as represented via a heat map. A convolution coefficient may be continuously computed for each scaled daughter wave (represented across a Y/Scale axis) and may be output continuously over time (represented on an X/Time axis. It should be appreciated that other wavelet-based analysis techniques exist (most commonly in the field of digital image compression) and may be used in combination with or instead of continuous or discrete wavelet transforms for the purposes described herein.

U.S. Pat. No. 1,850,332 (Shelton) evidences methods for treating tissue are provided. In one embodiment, an adjunct material, when secured to tissue, can receive at least one physiological element released from the tissue during healing progression of the tissue, and can exhibit first and second stiffnesses in compression that are approximately constant during first and second time periods from contact with the tissue, with the second stiffness decreasing with time as a function of at least one of oxidation, enzyme-catalyzed hydrolysis, and change of pH resulting from interaction with the at least one physiological element. In another embodiment, the adjunct can receive a unit volume of fluid that causes first and second portions of the adjunct to expand according to first and second expansion behaviors that differ from one another to apply different pressures to the tissue.

Various chemistry including pharmaceuticals and active agents may be considered for their complexity of administration and required sophistication in a caregiver during the procedure. Non-limiting examples of antimicrobial agents include Ionic Silver, Aminoglycosides, Streptomycin, Polypeptides, Bacitracin, Triclosan, Tetracyclines, Doxycycline, Minocycline, Demeclocycline, Tetracycline, Oxytetracycline, Chloramphenicol, Nitrofurans, Furazolidone, Nitrofurantoin, Beta-lactams, Penicillins, Amoxicillin, Amoxicillin+Clavulanic Acid, Azlocillin, Flucloxacillin, Ticarcillin, Piperacillin+tazobactam, Tazocin, Biopiper TZ, Zosyn, Carbapenems, Imipenem, Meropenem, Ertapenem, Doripenem, Biapenem, Panipenem/betamipron, Quinolones, Ciprofloxacin, Enoxacin, Gatifloxacin, Gemifloxacin, Levofloxacin, Lomefloxacin, Moxifloxacin, Nalidixic Acid, Norfloxacin, Sulfonamides, Mafenide, Sulfacetamide, Sulfadiazine, Silver Sulfadiazine, Sulfadimethoxine, Sulfamethizole, Sulfamethoxazole, Sulfasalazine, Sulfisoxazole, Bactrim, Prontosil, Ansamycins, Geldanamycin, Herbimycin, Fidaxomicin, Glycopeptides, Teicoplanin, Vancomycin, Telavancin, Dalbavancin, Oritavancin, Lincosamides, Clindamycin, Lincomycin, Lipopeptide, Daptomycin, Macrolides, Azithromycin, Clarithromycin, Erythromycin, Roxithromycin, Telithromycin, Spiramycin, Oxazolidinones, Linezolid, Aminoglycosides, Amikacin, Gentamicin, Kanamycin, Neomycin, Netilmicin, Tobramycin, Paromycin, Paromomycin, Cephalosporins, Ceftobiprole, Ceftolozane, Cefclidine, Flomoxef, Monobactams, Aztreonam, Colistin, and Polymyxin B.

Non-limiting examples of antifungal agents include Triclosan, Polyenes, Amphotericin B, Candicidin, Filipin, Hamycin, Natamycin, Nystatin, Rimocidin, Azoles, Imidazole, Triazole, Thiazole, Allylamines, Amorolfin, Butenafine, Naftifine, Terbinafine, Echinocandins, Anidulafungin, Caspofungin, Micafungin, Ciclopirox, and Benzoic Acid.

Non-limiting examples of antiviral agents include uncoating inhibitors such as, for example, Amantadine, Rimantadine, Pleconaril; reverse transcriptase inhibitors such as, for example, Acyclovir, Lamivudine, Antisenses, Fomivirsen, Morpholinos, Ribozymes, Rifampicin; and virucidals such as, for example, Cyanovirin-N, Griffithsin, Scytovirin, a-Lauroyl-L-arginine ethyl ester (LAE), and Ionic Silver.

Non-limiting examples of anti-inflammatory agents include non-steroidal anti-inflammatory agents (e.g., Salicylates, Aspirin, Diflunisal, Propionic Acid Derivatives, Ibuprofen, Naproxen, Fenoprofen, and Loxoprofen), acetic acid derivatives (e.g., Tolmetin, Sulindac, and Diclofenac), enolic acid derivatives (e.g., Piroxicam, Meloxicam, Droxicam, and Lornoxicam), anthranilic acid derivatives (e.g., Mefenamic Acid, Meclofenamic Acid, and Flufenamic Acid), selective COX-2 inhibitors (e.g., Celecoxib (Celebrex), Parecoxib, Rofecoxib (Vioxx), Sulfonanilides, Nimesulide, and Clonixin), immune selective anti-inflammatory derivatives, corticosteroids (e.g., Dexamethasone), and iNOS inhibitors.

Non-limiting examples of growth factors include those that are cell signaling molecules that stimulate cell growth, healing, remodeling, proliferation, and differentiation. Exemplary growth factors can be short-ranged (paracrine), long ranged (endocrine), or self-stimulating (autocrine). Further examples of the growth factors include growth hormones (e.g., a recombinant growth factor, Nutropin, Humatrope, Genotropin, Norditropin, Saizen, Omnitrope, and a biosynthetic growth factor), Epidermal Growth Factor (EGF) (e.g., inhibitors, Gefitinib, Erlotinib, Afatinib, and Cetuximab), heparin-binding EGF like growth factors (e.g., Epiregulin, Betacellulin, Amphiregulin, and Epigen), Transforming Growth Factor alpha (TGF-a), Neuroregulin 1-4, Fibroblast Growth Factors (FGFs) (e.g., FGF1-2, FGF2, FGF11-14, FGF18, FGF15/19, FGF21, FGF23, FGF7 or Keratinocyte Growth Factor (KGF), FGF10 or KGF2, and Phenytoin), Insuline-like Growth Factors (IGFs) (e.g., IGF-1, IGF-2, and Platelet Derived Growth Factor (PDGF)), Vascular Endothelial Growth Factors (VEGFs) (e.g., inhibitors, Bevacizumab, Ranibizumab, VEGF-A, VEGF-B, VEGF-C, VEGF-D and Becaplermin). Additional non-limiting examples of the growth factors include cytokines, such as Granulocyte Macrophage Colony Stimulating Factors (GM-CSFs) (e.g., inhibitors that inhibit inflammatory responses, and GM-CSF that has been manufactured using recombinant DNA technology and via recombinant yeast-derived sources), Granulocyte Colony Stimulating Factors (G-CSFs) (e.g., Filgrastim, Lenograstim, and Neupogen), Tissue Growth Factor Beta (TGF-B), Leptin, and interleukins (ILs) (e.g., IL-1a, IL-1b, Canakinumab, IL-2, Aldesleukin, Interking, Denileukin Diftitox, IL-3, IL-6, IL-8, IL-10, IL-11, and Oprelvekin). The non-limiting examples of the growth factors further include erythropoietin (e.g., Darbepoetin, Epocept, Dynepo, Epomax, NeoRecormon, Silapo, and Retacrit). Non-limiting examples of analgesics include Narcotics, Opioids, Morphine, Codeine, Oxycodone, Hydrocodone, Buprenorphine, Tramadol, Non-Narcotics, Paracetamol, acetaminophen, NSAIDS, and Flupirtine.

Non-limiting examples of anesthetics include local anesthetics (e.g., Lidocaine, Benzocaine, and Ropivacaine) and general anesthetic. Non-limiting examples of tissue matrix degradation inhibitors that inhibit the action of metalloproteinases (MMPs) and other proteases include MMP inhibitors (e.g., exogenous MMP inhibitors, hydroxamate-based MMP inhibitors, Batimastat (BB-94), Ilomastat (GM6001), Marimastat (BB2516), Thiols, Periostat (Doxycycline), Squaric Acid, BB-1101, Hydroxyureas, Hydrazines, Endogenous, Carbamoylphosphates, Beta Lactams, and tissue Inhibitors of MMPs (TIMPs)). Exemplary medicants also include agents that passively contribute to wound healing such as, for example, nutrients, oxygen expelling agents, amino acids, collageno synthetic agents, Glutamine, Insulin, Butyrate, and Dextran. Exemplary medicants also include anti-adhesion agents, non-limiting examples of which include Hyaluronic acid/Carboxymethyl cellulose (seprafilm), Oxidized Regenerated Cellulose (Interceed), and Icodextrin 4% (Extraneal, Adept). Exemplary medicants also include agents that encourage blood supply regeneration following coronary artery disease (CAD) (e.g., VEGF₁₆₅ protein, AdVEGF.sub.₁₆₅, AdVEGF₁₂₁, and VEGF₁₆₅ plasmid) or periphery artery disease (PAD) (e.g., VEGF₁₆₅ plasmid, AdVEGF₁₂₁, SB-509 (SFP-VEGF plasmid), AdVEGF₁₆₅, and Ad2-HIF1α-VP16 (WALK trial)).

### Drug Release

An adjunct (optional functional procedural element or component) in accordance with the described techniques can be associated with at least one medicant in a number of different ways, so as to provide a desired effect, such as on tissue in-growth, in a desired manner. The at least one medicant can be configured to be released from the adjunct in multiple spatial and temporal patterns to trigger a desired healing process at a treatment site. The medicant can be disposed within, bonded to, incorporated within, dispersed within, or otherwise associated with the adjunct. For example, the adjunct can have one or more regions releasably retaining therein one or more different medicants. The regions can be distinct reservoirs of various sizes and shapes and retaining medicants therein in various ways, or other distinct or continuous regions within the adjuncts. In some aspects, a specific configuration of the adjunct allows it to releasably retain therein a medicant or more than one different medicant.

Regardless of the way in which the medicant is disposed within the adjunct, an effective amount of the at least one medicant can be encapsulated within a vessel, such as a pellet which can be in the form of microcapsules, microbeads, or any other vessel. The vessels can be formed from a bioabsorbable polymer. Targeted delivery and release of at least one medicant from an adjunct can be accomplished in a number of ways which depend on various factors. In general, the at least one medicant can be released from the adjunct material as a bolus dose such that the medicant is released substantially immediately upon delivery of the adjunct material to tissue. Alternatively, the at least one medicant can be released from the adjunct over a certain duration of time, which can be minutes, hours, days, or more. A rate of the timed release and an amount of the medicant being released can depend on various factors, such as a degradation rate of a region from which the medicant is being released, a degradation rate of one or more coatings or other structures used to retain the medicant within the adjuncts, environmental conditions at a treatment site, and various other factors. In some aspects, when the adjunct has more than one medicant disposed therein, a bolus dose release of a first medicant can regulate a release of a second medicant that commences release after the first medicant is released. The adjunct can include multiple medicants, each of which can affect the release of one or more other medicants in any suitable way. Release of at least one medicant as a bolus dose or as a timed release can occur or begin either substantially immediately upon delivery of the adjunct material to tissue, or it can be delayed until a predetermined time. The delay can depend on a structure and properties of the adjunct or one or more of its regions.

An adjunct material can be configured to have a structure that facilitates distribution of effective amounts of one or more medicants carried within the adjunct to provide a desired effect. For example, the targeted delivery of the medicants can be accomplished by incorporating the medicants into regions (e.g., reservoirs such as pores or other structures) within the adjunct formed in a pattern that allows a certain spatial distribution of the medicants upon their delivery. The medicants disposed within the reservoir can be incorporated into distinct vessels. A reservoir can include more than one type of different medicants. The one or more medicants can be eluted from the adjunct in a homogeneous manner or in heterogeneous spatial and/or temporal manner to deliver a desired therapy. The structure of the adjunct and the way in which the medicants are released therefrom can be used to influence or control tissue re-growth. Moreover, the tissue regrowth can be encouraged in certain locations at the treatment site and discouraged at other locations at the treatment site.

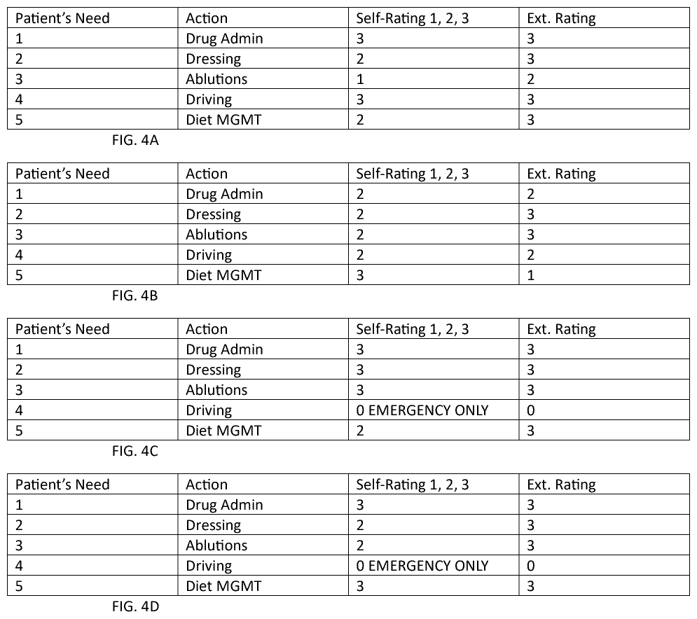

FIGS. 4A, 4B, 4C and 4D show individual potential caregiver scholastic rating charts.

Each of the tables 4A-4D show self ratings and an external rating on five services requested by the specific patient or generally reviewed by the operator of the central processor. Each table will have a specific price attached to it and information regarding potential financial assistance of coverage dependent upon the specific patient's needs and insurance coverage and local assistance programs.

As can be seen, certain features may not be available (e.g., casual driving), and other features may have higher or lower levels of skill needed than requested. Some categories may be adjusted in importance by the central processor without specific patient knowledge or requested input. For example, drug management can vary in importance beyond the knowledge of the specific patient. In some circumstances, little more than administration of OTC pain medications (e.g., acetaminophens, ibuprofen, aspirin) may be all that is needed, and the relative importance assessed by the specific patient may be out of line. In other circumstances, the patient may believe that this value is not important, even when there are more than 10 critical drugs being orally taken at different intervals and times of the day. The artificial intelligence of the central processor will step into these valuations and eliminate from consideration any table with a scholastic valuation of less than 3, or even a strong two (both self and external valuation) because of the importance in that individual circumstance.

In the above description, the use of “scholastic” measurements/estimates of functions and abilities and goals is used. Although the example is as simple ratings of 1, 2 or 3, the number values are not critical. Ranges of 0-3, 0-10, 1-10, or even letter grades of A, B, C . . . may be used. The ratings are subjective, and this is another reason why the use of artificial intelligence to sift through comparison of objective goals of needs and desires with the more subjective valuation of abilities and performance is desirable. The AI analysis is likely to direct weight and attention in the decisions into the direction of patient recovery needs rather than more aesthetic desires for comfort.

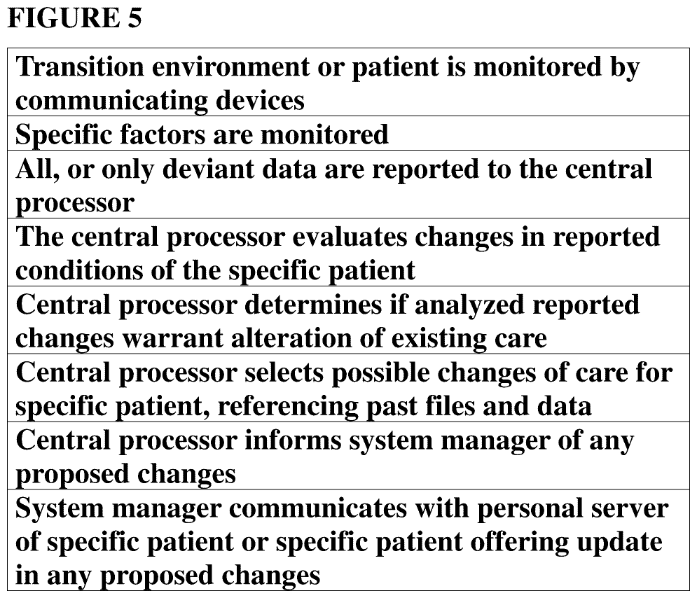

FIG. 5 shows a flow diagram/table for a procedure that follows-up monitoring of specific patient during and after transitioning of care. Monitoring systems are provided in the specific patient's care environment. These may be distal sensors, such as sound monitors, motion sensors, and the like, or be personally attached (long period or specific selected times). For example, health monitoring watch-like devices have become highly precise and can be used for direct and immediate (or longer collected data terms of days or weeks) sensing of medical data relating to the care of the specific patient and reporting of raw or highlighted data to the central processor.

The central processor receives sensed data such as pulse, blood pressure, heart rhythm, blood analysis (e.g., blood sugar, oxygen content), motion analysis (e.g., number of steps, frequency of activity, etc.), liquid intake, urine protein density or color (may be photographed by smart phone or camera and reported), skin coloration and mottling (again by photographic analysis), eye coloration (e.g., yellowing indicating organ issues), respiratory functions (again a phone or stethoscope can collect and transmit data), and specific patient input on subjective results of pain, tiredness and satisfaction with care.

The central processor executes software, comparing most recent results to recorded past results to evaluate the effects of current care levels and previous reported conditions for an analysis of the objective (sensed and reported) and subjective (specific patient reports on subject aspects of care satisfaction and conditions) effectiveness and evaluates the materiality of any changes, especially where alteration in care levels is determined by the central processor to be desirable or necessary.

Any resulting report of status of care is reported to the (real or virtual) case manager which then selects possible changes in the specific patient care and relays that information to the specific patient and the case worker (care provider) or the organization providing the care.

What is claimed:

1. A method is provided for improving extra-institutional care for a patient by:
a) Identifying a potential caregiver for a specific patient prior to transitioning from institutional care to home care;
b) Establishing an online communication link between a central processor and a processor accessible by the potential caregiver;
c) The central processor providing a set of questions to the processor accessible by the potential caregiver, the questions including queries concerning knowledge of and capability of providing the specific patient's conditions, the questions selected from the group consisting of i) medical needs, ii) physical needs, iii) emotional needs, iv) the potential caregiver's physical and intellectual ability to serve the specific patient, v) the potential caregiver's knowledgeability and comfort level in serving the specific patient, and vi) availability of at least one of equipment, location and accommodations desirable for the care of the specific patient;
d) The central processor receiving responses to the questions in a format that can be read as or converted to scholastic values for the responses;
e) The central processor executing software to characterize a level of scholastic performance within categories of the questions and responses;
f) The central processor selecting at least one stored caregiver plan consistent with the characterized level of scholastic performance within the categories of the questions and responses;
g) A case manager having access to the central computer and the at least one stored caregiver plan communicating with the potential caregiver to relay and explain details of the at least one stored caregiver plan prior to transfer of the specific patient to care by the potential caregiver.

2. The method of claim 1 wherein the central processor identifies more than one stored caregiver plans, and ranks the more than one stored caregiver plans according to closest matches with needs indicated by the scholastic values attributed to the responses of the potential caregiver.

3. The method of claim 2 wherein the central processor also reviews stored medical history of the specific patient and adjusts weighting of the scholastic values attributed to the responses of the potential caregiver in according with programmed importance of responses with respect to specific medical needs of the specific patient in accordance with proposed medical treatment for the specific patient.

4. The method of claim 1 wherein the at least one stored caregiver plan includes providing information relating to at least one resource selected from the group consisting of A) available government and non-governmental services for the specific patient, B) available support personnel, C) online or telephonic information regarding patient care, and D) proprietary and open access materials.

5. The method of claim 1 wherein the specific patient and/or the potential caregiver is given a written or oral examination to determine a level of skill relative to implementing and overseeing administration of prescribed medication for the specific patient.

6. The method of claim 1 wherein when more than stored caregiver plan is offered to the specific patient, the specific patient elects a single stored caregiver plan for use.

7. The method of claim 1 wherein the central processor reviews caregiver policies from business entities and, based upon execution of software identifying best fits for available caregiver services, offers at least one caregiver plan to the specific patient.

8. The method of claim 7 wherein the processor offers a selection of multiple caregiver plans which are ranked according to at least one parameter selected from the group consisting of cost, relative match with patient needs, and ability of the caregiver plans to adjust over a time of treatment for the specific patient.

9. A system for enabling the method of claim 1, the system including the central processor and the processor accessible by the potential caregiver linked through a wireless connection, the central processor including a memory storage element programmed to execute software performing steps c). d), e) and f).

10. A system for enabling the method of claim 2, the system including the central processor and the processor accessible by the potential caregiver linked through a wireless connection, the central processor including a memory storage element programmed to execute software performing steps c). d), e) and f).

11. A system for enabling the method of claim 3, the system including the central processor and the processor accessible by the potential caregiver linked through a wireless connection, the central processor including a memory storage element programmed to execute software performing steps c). d), e) and f).

12. A system for enabling the method of claim 4, the system including the central processor and the processor accessible by the potential caregiver linked through a wireless connection, the central processor including a memory storage element programmed to execute software performing steps c). d), e) and f).

13. A system for enabling the method of claim 5, the system including the central processor and the processor accessible by the potential caregiver linked through a wireless connection, the central processor including a memory storage element programmed to execute software performing steps c). d), e) and f).

14. A system for enabling the method of claim 5, the system including the central processor and the processor accessible by the potential caregiver linked through a wireless connection, the central processor including a memory storage element programmed to execute software performing steps c). d), e) and f).

15. A system for enabling the method of claim 6, the system including the central processor and the processor accessible by the potential caregiver linked through a wireless connection, the central processor including a memory storage element programmed to execute software performing steps c). d), e) and f).

16. A system for enabling the method of claim 7, the system including the central processor and the processor accessible by the potential caregiver linked through a wireless connection, the central processor including a memory storage element programmed to execute software performing steps c). d), e) and f).

17. A system for enabling the method of claim 8, the system including the central processor and the processor accessible by the potential caregiver linked through a wireless connection, the central processor including a memory storage element programmed to execute software performing steps c). d), e) and f).
